(19) United States
(12) Patent Application Publication
(10) Pub. No.: US 2011/0106136 A1
(43) Pub. Date: May 5, 2011

(54) IVC Filter With Drug Delivery

(75) Inventors: Dustin Thompson, Santa Rosa, CA (US); D.H. Perkins, Santa Rosa, CA (US)

(73) Assignee: Medtronic Vascular, Inc., Santa Rosa, CA (US)

(21) Appl. No.: 12/608,217

(22) Filed: Oct. 29, 2009

(51) Int. Cl. A61F 2/01

(52) U.S. Cl. 606/200

(57) ABSTRACT

Retrievable inferior vena cava filters are disclosed. Such filters provide for easy removal of the filter and provide drug delivery for dissolution of captured emboli. Methods of using such systems for the prevention, capture, dissolution and removal of emboli are described.

### FIELD OF THE INVENTION

The present invention relates to filters within a vessel. In particular, the present invention relates to retrievable vena cava filters that are easily removable and employ means for preventing, capturing, dissolving and removing trapped emboli.

### BACKGROUND OF THE INVENTION

Inferior Vena Cava (IVC) filters are currently used to prevent venous emboli from migrating through the heart to the lungs, resulting in a pulmonary embolism (PE). Pulmonary embolism is a blockage of the pulmonary artery by a blood clot, or thrombus. In ninety percent of cases, these blood clots originate in the lower extremity and travel through the inferior vena cava before passing through the right side of the heart and entering the lungs. In twenty-five percent of cases, the extent of the embolism leads to sudden death. Patients at risk for PE are trauma, surgical (e.g. hip replacement or spine repair), cancer, venous disease (including deep vein thrombosis and chronic venous insufficiency) patients. The current standard of care for PE patients, or patients at risk of a PE, is anticoagulation therapy. Patients contraindicated for anticoagulation therapy, due to bleeding risks, or those at high risk of an initial or recurrent PE are treated with an IVC filter. The devices are generally implanted within the IVC and function by capturing emboli contained in the blood stream before they can reach the lungs and cause permanent damage to the patient. They generally employ a series of legs or other features that are expanded in the vessel to form a conical-shaped filtering surface on which emboli are collected. To anchor the filter in the vessel and prevent it from migrating to the heart, hooks, barbs or similar piercing means on the legs are employed. IVC filters implanted for an indefinite amount of time, deemed permanent, may also result in a permanent obstruction in the vessel and result in dangerous disruption of normal hemodynamic flow. Furthermore, the use of systemic anticoagulant and/or thrombolytic agents presents additional risk to the patient due to the high systemic dosage of such agents that is needed to dissolve captured emboli. While IVC filters were originally designed as permanent implants, more recently there has been a focus on retrievable filters that can be removed once the threat of PE has passed. For example, an IVC filter need only be in place during and for a short time after certain medical and surgical procedures that carry a significant risk of PE. One concern with retrievable IVC filters is how to safely remove captured emboli.

Accordingly, it would be desirable to have an IVC filter that can be implanted in a patient at risk for PE and is easily removable once the risk has passed without risk or damage to the patient, and where emboli are captured, dissolved, or otherwise safely removed without the need for systemic anticoagulant or thromblytic agents.

### SUMMARY OF THE INVENTION

The present invention relates to IVC filters that allow for temporary implantation of an IVC filter that is easily removed. The invention also relates to IVC filters having drug delivery capability.

According to the teachings of the present invention there is provided a filter positionable within a blood vessel for trapping and removing emboli comprising an apical head; a plurality of divergent legs each leg secured at one of its ends to said head, said legs comprising a hollow tube having a wall, a lumen and a plurality of openings completely through the wall and communicating with the lumen, each leg having an anchor member at the distal end thereof with respect to said head, said anchor member arranged outwardly from said leg; an anticoagulant or thrombolytic agent disposed within the lumen; and a retrieval member operably attached to said head.

According to further teachings of the present invention there is provided a method of capturing, dissolving and removing emboli from a blood vessel of a patient comprising delivering with a delivery system a filter to a target location in the blood vessel, the filter comprising an apical head; a plurality of divergent legs each leg secured at one of its ends to said head, said legs comprising a hollow tube having a wall, a lumen and a plurality of openings completely through the wall and communicating with the lumen, each leg having an anchor member at the distal end thereof with respect to said head, said anchor member arranged outwardly from said leg; an anticoagulant or thrombolytic agent disposed within the lumen; and a retrieval member operably attached to said head; leaving the filter in place for a determined period of time to prevent, capture and dissolve emboli; snaring the retrieval member of the filter; and removing the filter from the patient.

### BRIEF DESCRIPTION OF THE DRAWINGS

FIG. 1 is a schematic view of an IVC filter of the present invention.

FIG. 2 is a radial cross section of a leg of filter of FIG. 1. taken along line 2-2.

FIG. 3 is a portion of a leg of another embodiment of a filter of the present invention.

FIG. 4 is a flowchart depicting a method of capturing and removing emboli.

### DETAILED DESCRIPTION OF THE INVENTION

The present invention relates to inferior vena cava (IVC) filters and methods. In particular, the present invention relates to IVC filters that allow for a filter to be temporarily implanted and easily removed. The filter has capability for infusing an anticoagulant agent and/or a thrombolytic agent out of openings in the wall of the filter. The invention also relates to methods for utilizing the aforementioned IVC filter for the capture, dissolution and removal of emboli.

The principles and operation of devices and methods according to the present invention may be better understood with reference to the drawings and the accompanying description, where like reference numerals refer to like elements.

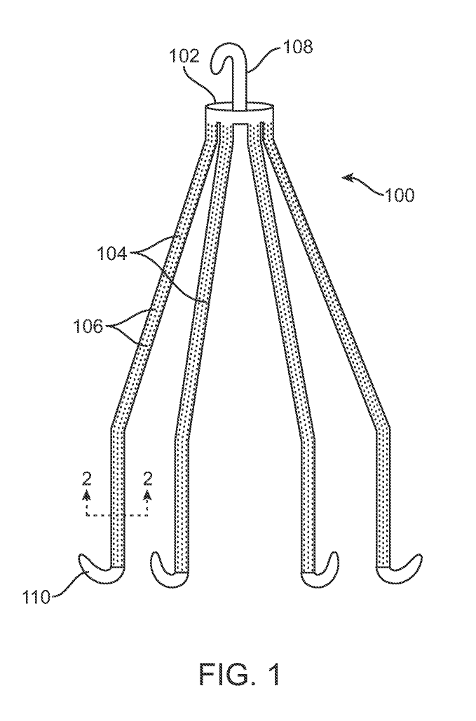

With reference to FIG. 1 a typical embodiment of an IVC filter 100 of the present invention is illustrated. The filter has an apical head 102, a plurality of divergent legs 104, each leg formed of a hollow tube and attached at one end to the apical head. Each leg has an anchor member 110 at the distal end thereof with respect to the apical head. A retrieval member 108 is operably attached to the apical head. The legs have a plurality of openings 106 through the wall that communicate with the lumen. In one embodiment, the openings are spaced along the hollow legs in fluid communication with the lumen of the hollow legs. In another embodiment, the openings are located at the ends of the hollow legs.

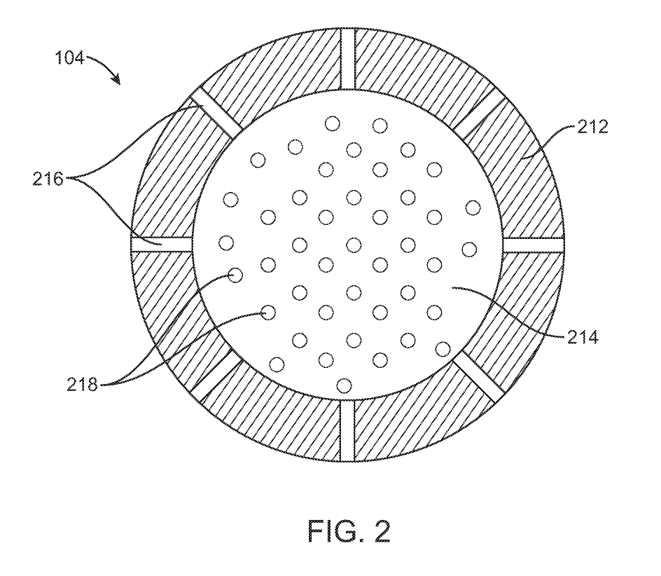

With reference to FIG. 2 a radial cross section of a typical tube of leg 104 taken along line 2-2 of the filter of FIG. 1 is depicted. The tube has a wall 212, a lumen 214, a plurality of openings 216 completely through the wall and communicating with the lumen and a therapeutic agent 218 disposed in the lumen. In one embodiment, therapeutic agent 218 comprises an anticoagulant or thrombolytic agent. The anticoagulation agents and thrombolytic agents function to assist in the prevention of emboli formation and/or dissolution of captured emboli, respectively. Suitable anticoagulant agents are well known in the art and include, for example, heparin, coumadin, aspirin, ticlopidine, clopidogrel and prasugrel. Suitable thrombolytic agents are well known in the art and include, for example, tPA, reteplase, alteplase, tenecteplase, activase, lanoteplase, staphylokinase, streptokinase and urokinase. Lumen 214 may contain other therapeutic agents or combinations of agents.

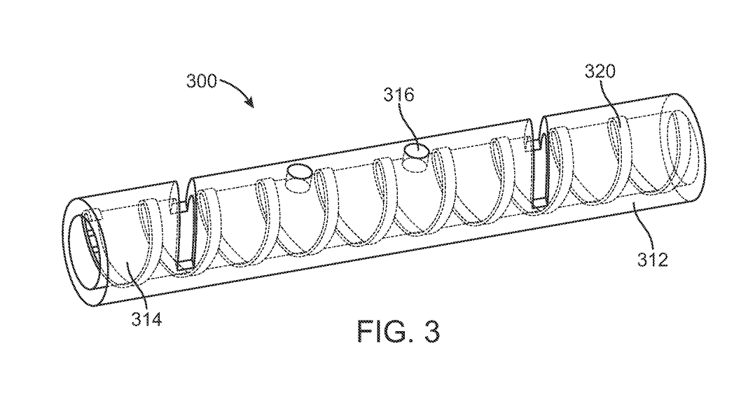

The legs 104 are biased outward so that when the filter is deployed the legs expand outwardly and are divergent as depicted in FIG. 1. The legs are comprised of a hollow tube that may be constructed from any number of materials such as hollow polymeric fibers or hollow metallic fibers such as stainless steel alloys, cobalt chromium alloys (e.g. MP35N, Conichrome®, Phynox® and Elgiloy®), nickel titanium alloys (e.g. Nitinol), etc. The tube is preferably comprised of a superelastic material such as Nitinol or a spring temper stainless steel or cobalt chromium alloy. Alternatively, the tube may be a composite, for example, a polymeric tube having a wire coil wound within to provide greater structural integrity, as discussed below for FIG. 3. Suitable polymers that may be used for such tubes include, for example, ePTFE, silicones, polyethylenes, high-density polyethylenes, low-density polyethylenes, polyimides, PEBAX, etc. The tubes would typically have a diameter between about 0.10 mm and 1.50 mm, with a lumen diameter of between about 0.05 mm and 1.25 mm. The tubes have a plurality of openings 216 therein, such openings being completely through the wall 212 and communicating with the lumen 214. In the embodiment depicted in FIG. 1 the openings are shown as being substantially circular. Preferred sizes for such circular openings are between about 5 and about 40 microns. However, one skilled in the art will recognize that such openings may be of various shapes and sizes depending upon the specific situation such as the nature and formulation of anticoagulant or thrombolytic agent being eluted. For example, the openings may be oval, square or rectangular in shape or may form elongated channels.

Attached to the distal ends of the leg is anchor member 110 used to secure the legs of the filter to the wall of the blood vessel. The anchor member is arranged outwardly from the leg, typically at an acute angle thereto, so that it engages with the wall of the blood vessel when the filter is deployed. Typically the anchoring member comprises a sharp tip or similar piercing element to penetrate the wall of the blood vessel.

Retrieval member 108 is operably attached to the apical head 102. The retrieval member may be any element that allows the filter to be captured so that it can be removed from the patient's body. In FIG. 1 the retrieval member is shown as having a hook shape. It will be apparent to one skilled in the art that other shapes may be employed, for example, an eyelet that would be captured by a hook or other grasping means such as a clamp, pincher, etc.

FIG. 3 depicts a composite tube 300 used for a leg of another embodiment of the filter. The composite tube has a polymer jacket 312, having a wound metal coil 320 disposed in the lumen thereof for structural support. Openings 316 through the polymer jacket communicate with the lumen 314 of the tube. The openings may have various shapes such as round, oval, square or rectangular, or may form elongated channels. The openings may be of sizes as described above with reference to FIG. 1.

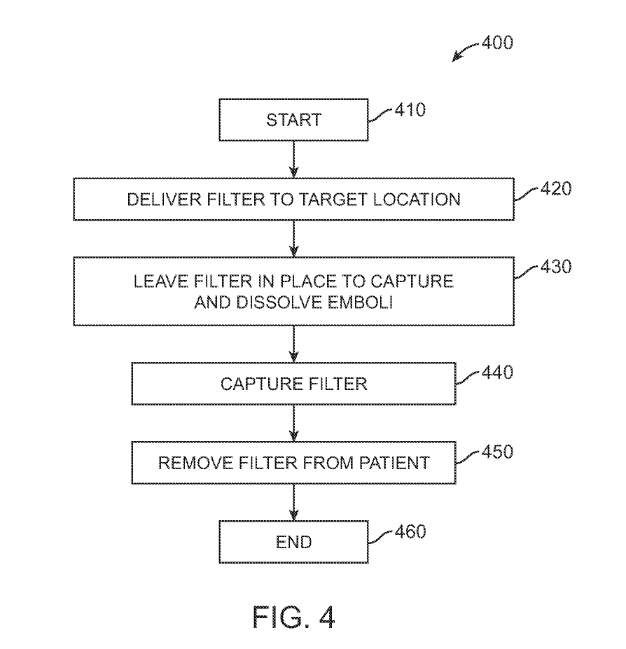

FIG. 4 represents a flowchart depicting the various steps in a method 400 of capturing and removing venous emboli from a patient.

Method 400 begins at step 410. At step 420 the filter is delivered to a target location in the blood vessel using a delivery system. Typically, a guidewire is delivered to a target location in the inferior vena cava of a patient. The inferior vena cava is accessed intravascularly through one of a number of veins such as the femoral vein, the jugular vein or the subclavian vein. A delivery catheter containing the filter is advanced over the guidewire to the target location and the filter is deployed by pushing it out of the catheter, pulling back on the catheter relative to the filter, or removing a sheath constraining the filter.

At step 430 the filter is left in place for a determined period of time to allow the anticoagulant or thrombolytic agent to elute therefrom through the openings to prevent, capture and dissolve emboli.

At step 440 the filter is captured on its retrieval member 108 by engaging with a capture member advanced through a catheter. The capture member and the retrieval member are complementary, such that they engage each other. For example, if the retrieval member is a hook, the capture member may be an eyelet, and vice-versa. Intravascular snare devices that may be employed to capture the retrieval member 108 are well known in the art and include, for example, the Amplatz GOOSE NECK Snare, manufactured by ev3, Plymouth Minn.

At step 450 the filter is removed from the patient by withdrawing the capture member having the filter engaged thereto through a catheter, during which process the filter legs are collapsed inwardly to allow for the filter to fit inside the lumen of the catheter.

Method 400 ends at step 460.

Having thus described the several embodiments of the present invention, those of skill in the art will readily appreciate that other embodiments may be made and used that fall within the scope of the appended claims. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of this invention.

What is claimed is:

1. A filter positionable within a blood vessel for trapping and removing emboli comprising
a. an apical head;
b. a plurality of divergent legs each leg secured at one of its ends to said head, said legs comprising a hollow tube having a wall, a lumen and a plurality of openings completely through the wall and communicating with the lumen, each leg having an anchor member at the distal end thereof with respect to said head, said anchor member arranged outwardly from said leg;
c. an anticoagulant or thrombolytic agent disposed within the lumen; and
d. retrieval member operably attached to said head.

2. The filter of claim 1 wherein the legs comprise a superelastic material.

3. The filter of claim 2 wherein the superelastic material is nitinol.

4. The filter of claim 1 wherein the legs comprise a spring temper metallic material.

5. The filter of claim 1 wherein the legs comprise a composite structure.

6. The filter of claim 5 wherein the composite structure is a polymer tube surrounding a wire coil.

7. The filter of claim 1 wherein the openings are substantially circular.

8. The filter of claim 7 wherein the substantially circular openings have a diameter between about 5 and about 40 microns.

9. The filter of claim 1 wherein said anchor member is arranged at an acute angle with respect to the leg so as to engage with the wall of the vessel when deployed.

10. The filter of claim 1 wherein the anticoagulant agent is selected from the group consisting of heparin, coumadin, aspirin, ticlopidine, clopidogrel and prasugrel, and the thrombolytic agent is selected from the group consisting of tPA, reteplase, alteplase, tenecteplase, activase, lanoteplase, staphylokinase, streptokinase and urokinase.

11. A method of capturing and removing emboli from a blood vessel of a patient comprising
a. delivering with a delivery system a filter to a target location in the blood vessel, the filter comprising
i. an apical head;
ii. a plurality of divergent legs each leg secured at one of its ends to said head, said legs comprising a hollow tube having a wall, a lumen and a plurality of openings completely through the wall and communicating with the lumen, each leg having an anchor member at the distal end thereof with respect to said head, said anchor member arranged outwardly from said leg;
iii. an anticoagulant or thrombolytic agent disposed within the lumen; and
iv. a retrieval member operably attached to said head;
b. leaving the filter in place for a determined period of time to prevent, capture and dissolve emboli;
c. capturing the filter; and
d. removing the filter from the patient.

12. The method of claim 11 wherein the anticoagulant agent is selected from the group consisting of heparin, coumadin, aspirin, ticlopidine, clopidogrel and prasugrel, and the thrombolytic agent is selected from the group consisting of tPA, reteplase, alteplase, tenecteplase, activase, lanoteplase, staphylokinase, streptokinase and urokinase.
